(19) United States
(12) Patent Application Publication
(10) Pub. No.: US 2010/0210947 A1
(43) Pub. Date: Aug. 19, 2010

(54) Vascular Flow Sensor With Acoustic Coupling Detector

(75) Inventors: Michael Burcher, Tarrytown, NY (US); Balasundar Raju, Tarrytown, NY (US)

Correspondence Address: PHILIPS INTELLECTUAL PROPERTY & STANDARDS, P.O. BOX 3001, Briarcliff Manor, NY, 10510-8001, US

(73) Assignee: Koninklijke Philips Electronics N.V., Eindhoven (NL)

(21) Appl. No.: 12/085,132

(22) PCT Filed: Nov. 10, 2006
(86) PCT No.: PCT/IB2006/054200

§ 371 Date: May 15, 2008

Related U.S. Application Data
(60) Provisional application No. 60/737,911, filed on Nov. 17, 2005.
(60) Provisional application No. 60/782,996, filed on Mar. 16, 2006.

(51) Int. Cl. A61B 8/06

(52) U.S. Cl. 600/454

(57) ABSTRACT

A vascular flow sensor assembly includes an ultrasound transducer (1-5) which is attached to the body by an adhesive or mechanical substrate to create acoustic coupling between the ultrasound transducer and the body. The assembly includes a force sensor (150) responsive to the attachment force which produces a signal representative of the force of attachment. The signal is detected by a monitoring instrument and used to alert a user to improper attachment of the vascular sensor. In an illustrated embodiment the signal actuates an indicator located on the sensor assembly.

This invention relates generally to the field of cardiac resuscitation and, more specifically, to a sensor which detects vascular blood flow and tissue motion by ultrasonic Doppler techniques.

In emergencies and during operative procedures, the assessment of the state of blood flow of the patient is essential for both diagnosis of the problem and determining the appropriate therapy for the problem. The presence of a cardiac pulse in a patient is typically detected by palpating the patient's neck and sensing palpable pressure changes due to the change in the patient's carotid artery volume. When the heart's ventricles contract during a heartbeat, a pressure wave is sent throughout the patient's peripheral circulation system. A carotid pulse waveform rises with the ventricular ejection of blood at systole and peaks when the pressure wave from the heart reaches a maximum. The carotid pulse falls off again as the pressure subsides toward the end of the pulse.

The absence of a detectable cardiac pulse in a patient is a strong indicator of cardiac arrest. Cardiac arrest is a life-threatening medical condition in which the patient's heart fails to provide blood flow to support life. During cardiac arrest, the electrical activity of the heart may be disorganized (ventricular fibrillation), too rapid (ventricular tachycardia), absent (asystole), or organized at a normal or slow heart rate without producing blood flow (pulseless electrical activity).

The form of therapy to be provided to a patient without a detectable pulse depends, in part, on an assessment of the patient's cardiac condition. For example, a caregiver may apply a defibrillation shock to a patient experiencing ventricular fibrillation (VF) or ventricular tachycardia (VT) to stop the unsynchronized or rapid electrical activity and allow a perfusing rhythm to return. External defibrillation, in particular, is provided by applying a strong electric shock to the patient's heart through electrodes placed on the surface of the patient's chest. If the patient lacks a detectable pulse and is experiencing asystole or pulseless electrical activity (PEA), defibrillation cannot be applied and the caregiver may perform cardiopulmonary resuscitation (CPR), which causes some blood to flow in the patient.

Before providing therapy such as defibrillation or CPR to a patient, a caregiver must first confirm that the patient is in cardiac arrest. In general, external defibrillation is suitable only for patients that are unconscious, apneic, pulseless, and in VF or VT. Medical guidelines indicate that the presence or absence of a cardiac pulse in a patient should be determined within 10 seconds. For example, the American Heart Association protocol for cardiopulmonary resuscitation (CPR) requires a healthcare professional to assess the patient's pulse within five to ten seconds. Lack of a pulse is an indication for the commencement of external chest compressions. Assessing the pulse, while seemingly simple on a conscious adult, is the most often failed component of a basic life support assessment sequence, which may be attributed to a variety of reasons, such as lack of experience, poor landmarks, or error in either finding or not finding a pulse. Failure to accurately detect the presence or absence of the pulse will lead to adverse treatment of the patient either when providing or not providing CPR or defibrillation therapy to the patient.

Electrocardiogram (ECG) signals are normally used to determine whether or not a defibrillating shock should be applied. However, certain rhythms that a rescuer is likely to encounter cannot be determined solely by the ECG signal, e.g., pulseless electrical activity. Diagnoses of these rhythms require supporting evidence of a lack of perfusion despite the myocardial electrical activity as indicated by the ECG signal.

Thus, in order for a rescuer to quickly determine whether or not to provide therapy to a patient, it is necessary to develop an integrated system that is quickly and easily able to analyze the patient's pulse, the amount of blood flow, and perhaps the ECG signals in order to correctly determine whether there is any pulsatile flow in the arteries of the patient.

This necessity is particularly dire in situations or systems in which the rescuer is untrained and/or inexperienced person, as is the case with rescuers for which the system described in U.S. Pat. No. 6,575,914 (Rock et al.) is designed. The '914 patent is assigned to the same assignee as the present invention and is hereby incorporated by reference in its entirety. The '914 patent discloses an Automated External Defibrillator (AED) (hereinafter both AEDs and Semi-Automated External Defibrillators—SAEDs—will be referred to jointly as AEDs) which can be used by first-responding caregivers with little or no medical training to determine whether or not to apply defibrillation to an unconscious patient.

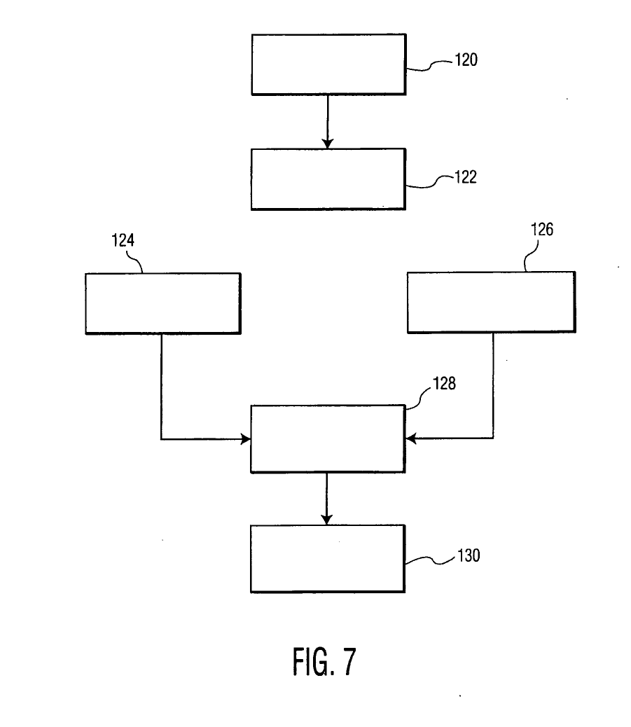

The Rock AED has a defibrillator, a sensor pad for transmitting and receiving Doppler ultrasound signals, two sensor pads for obtaining an ECG signal, and a processor which receives and assesses the Doppler and ECG signals in order to determine whether defibrillation is appropriate for the patient (i.e., whether or not there is a pulse) or whether another form of treatment such as CPR is appropriate. The Doppler pad is adhesively secured to a patient's skin above the carotid artery to sense the carotid pulse, which is a key indicator of the sufficiency of pulsatile blood flow. Specifically, the processor in the Rock AED analyzes the Doppler signals to determine whether there is a detectable pulse and analyzes the ECG signals to determine whether there is a “shockable rhythm.” See, e.g., FIG. 7 and accompanying description at col. 6, line 60, to col. 7, line 52, in the '914 patent. The determination of a detectable pulse by the processor in the Rock AED is made by comparing the received Doppler signals against a threshold statistically appropriate with the Doppler signals received. Based on the results of these two separate analyses, the processor determines whether or not to advise defibrillation.

U.S. patent application Ser. No. 60/583,966 filed Jun. 29, 2004 and now filed as international application IB 2005/052127 describes a Doppler pad similar to that of Rock which contains a plurality of ultrasound transducers that are multiplexed to detect vascular blood flow and measure blood flow characteristics such as perfusion, heart beat, tissue movement, flow of a colloidal or emulsion solution and the like. Concurrently filed US patent application [attorney docket 002603] describes a similar Doppler pad that measures vascular blood flow to assist with CPR coaching. In order to perform successfully it is important that a good acoustic path be established between the pad and the body. In particular, if the pad becomes loose or dislodged from secure contact with the skin surface the transmitted and/or received ultrasound waves can be severely attenuated and impede successful ultrasound echo detection. It is desirable that such undesired pad contact be detected and the user advised to correct the poor acoustical contact with the body.

In accordance with the principles of the present invention an ultrasonic transducer sensor is attached over the carotid artery or other blood vessel and used to sense the velocity of blood movement in the vessel. The transducer sensor assembly includes a force sensor which senses the force or tension of the attachment. The force or tension signal is detected by a monitoring instrument and used to alert a user to improper attachment of the transducer sensor, thereby alerting the user of disruption of the acoustic path of the transducer.

In the drawings:

FIG. 1 illustrates an ultrasonic sensor strip constructed in accordance with the principles of the present invention.

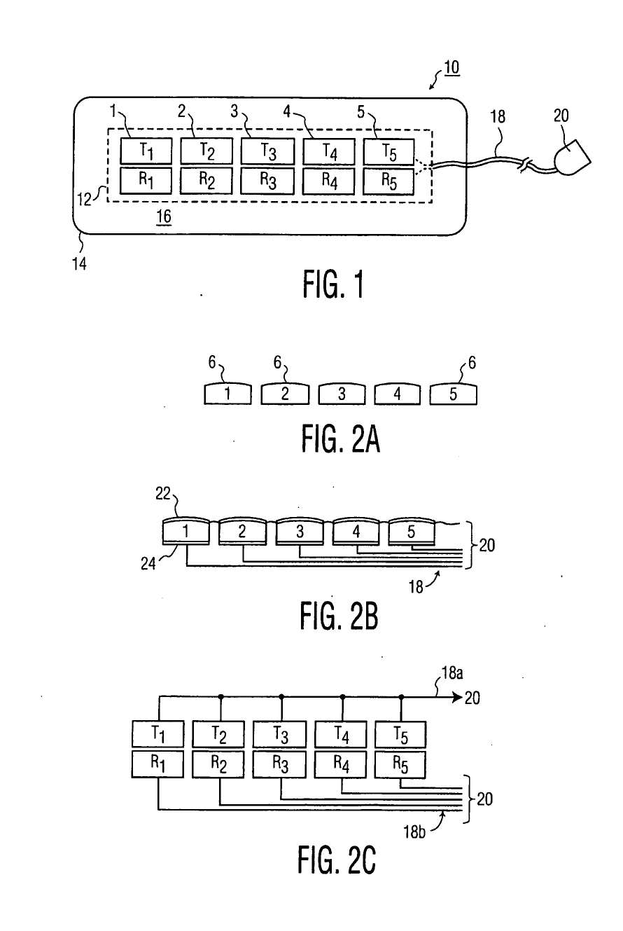

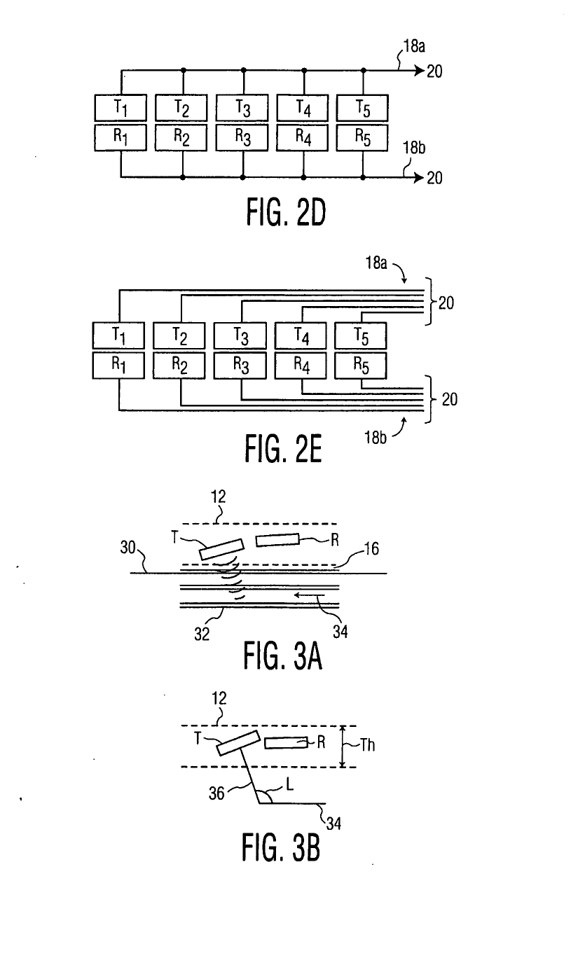

FIGS. 2a-2e illustrate different characteristics and configurations of the transducers of an ultrasonic sensor strip.

FIGS. 3a-3b illustrate the inclination of the transducers of an ultrasonic sensor strip.

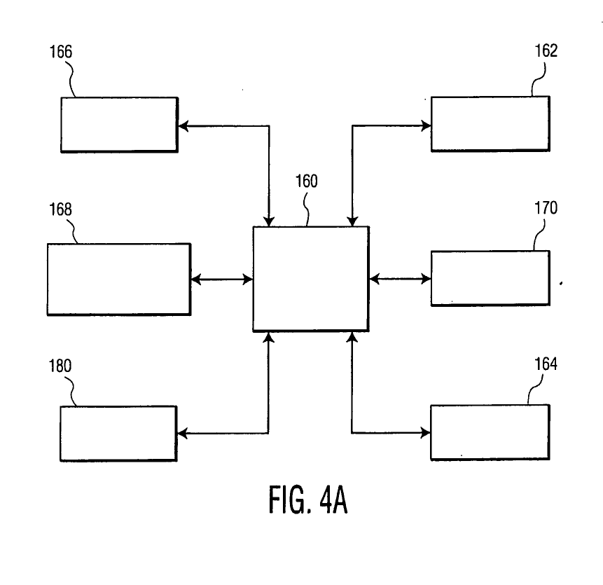

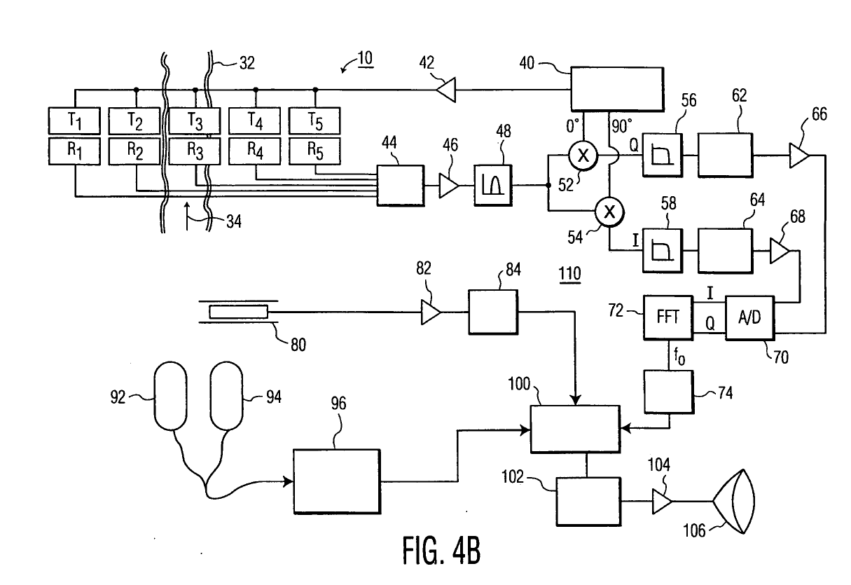

FIG. 4a illustrates in block diagram form a vital signs monitor and therapy system constructed in accordance with the principles of the present invention.

FIG. 4b illustrates in block diagram form a portion of a vital signs monitor and therapy system with pulse detection and CPR guidance constructed in accordance with the principles of the present invention.

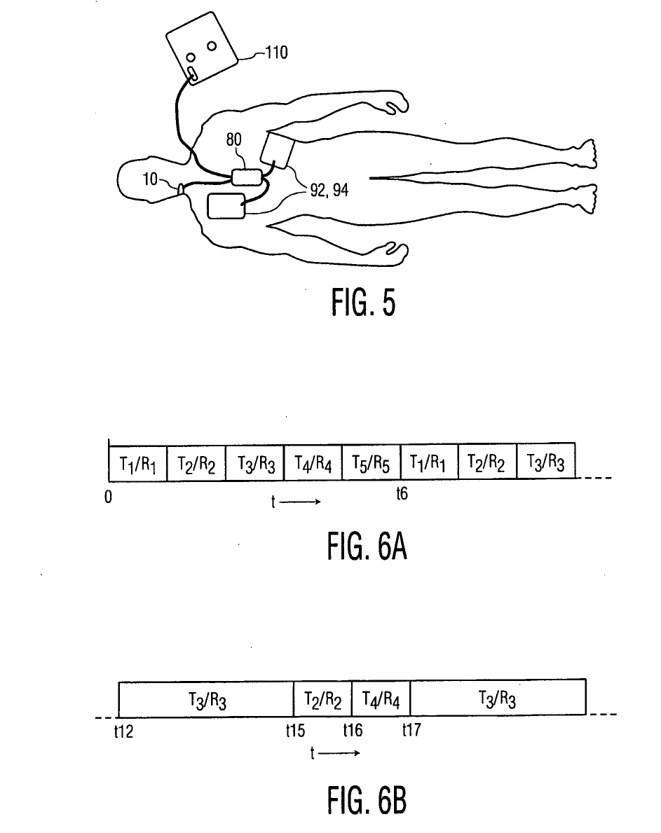

FIG. 5 illustrates application of the electrode pads and sensors of the defibrillator system of FIG. 4.

FIGS. 6a-6b illustrate one example of sequential operation of the transducers of an ultrasonic sensor strip.

FIGS. 7-9 illustrate three procedures which integrate blood flow sensing with other measured parameters for CPR guidance.

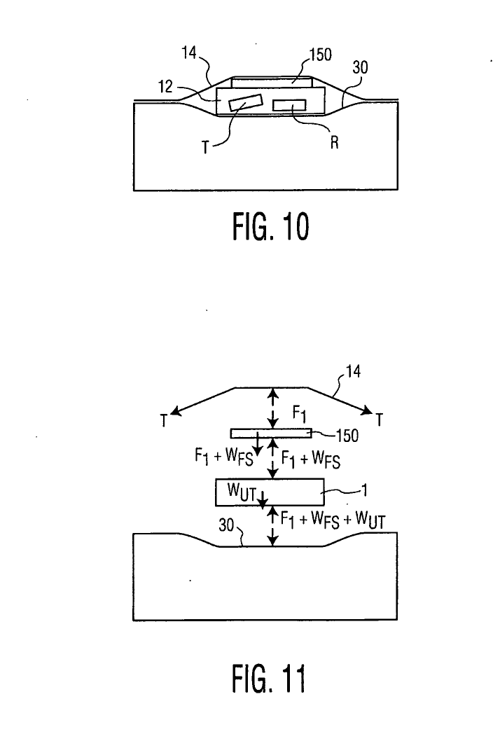

FIGS. 10-14 illustrate several examples of an ultrasonic sensor strip with a force sensor and indicator to provide an indication of the adequacy of acoustic coupling between the ultrasound transducers and the patient's body.

Referring first to FIG. 1, an ultrasonic sensor strip 10 constructed in accordance with the principles of the present invention is shown. The sensor strip 10 includes a row of pairs of transducers 1-5. Any number of transducers can be used in a given sensor strip with the number generally being in the range of four to six transducers. Each pair of transducer elements includes a transmitting element (T₁, T₂, etc.) and a receiving element (R₁, R₂, etc.) which enables operation in a continuous wave (CW) ultrasound mode: while the transmitting element is transmitting a wave, the corresponding receiving element is receiving echoes returned in response to the transmission. In this example the transducer elements are unfocussed and individually collimated with a cross-over at a depth of 1.5-2 cm and a range of 0.5-4 cm over which the apertures of the transmit and receive beams overlap so that echoes produced by a transmit transducer element will be received by the corresponding receive transducer element. For pulsed wave (PW) ultrasound operation only a single element is needed which sequentially transmits then receives. The transducers are enclosed in a flexible matrix 12 which can bend to conform to the shape of the skin surface to which the strip is applied. The transducers in the illustrated example are separated by a distance of 1-2 mm so that the row of transducers in the matrix can be bent. The matrix 12 maintains the alignment of the transducers and provides electrical insulation from the body and may be made of silicone or RTV rubber, for instance. Extending from the matrix 12 is a cable 18 of electrical conductors coupled to the transducer elements as described below. The cable 18 terminates at a connector which connects to a monitoring instrument with which the sensor strip 10 operates. The matrix of transducers is covered by a substrate 14 which adheres the sensor strip to the body. The sensor strip may be attached to the body by an elastic band, a necklace or a Velcro strap. In the illustrated example the substrate is an adhesive tape or other natural or polymeric material which has an adhesive 16 on its skin-contacting surface. The skin-contacting surface of the matrix of transducers is covered with a material which provides good acoustic coupling between the matrix 12 and the body. This acoustic material may be the same material as adhesive 16 when adhesive 16 has the desired acoustic properties such as an adhesive electrode gel material. The acoustic material may alternately comprise a hydrogel material or an adhesive patch or other solid material.

FIG. 2a is a side view of an example of transducers 1-5. In this example it is seen that the top transmitting surfaces 6 of the transducer elements are rounded. In this example the transducer elements are curved with a 25 mm radius of curvature. The rounding of the transmitting surfaces causes the emitted ultrasound to diverge and thereby insonify a greater area of the body, increasing the likelihood that a target vessel will be insonified and preventing any dead zones between the transducer elements. As an alternative to rounding the shape of the transducer a lens may be used above a flat emitting surface to cause the emitted ultrasound to diverge.

FIG. 2b shows electrical connections made to the transducers 1-5. The transmitting surfaces of the transducer elements which face the skin are covered with an electrode 22 which is grounded for safety. Individual electrodes 22 may be formed on the individual elements which are then electrically connected to the connector 20 by way of cable 18. Alternately the electrode 22 may be a continuous sheet of foil or other flexible, conductive material which covers groups or all of the transducer elements. The sides of the elements which face away from the skin surface have signal electrodes 24 on them. Conductors of cable 18 are connected to these electrodes 24 to provide transmit (drive) signals and return received echo signals from the transducer elements. FIG. 2c is a plan view of the transducer elements showing one example of connection of the signal conductors. In this example all of the transmit elements T₁-T₅ are operated together and electrically connected to one conductor 18a of the cable. The receive elements R₁-R₅ are operated separately and are connected to individual conductors 18b of the cable. This configuration enables all of the transmitting elements to be driven simultaneously by the same transmit wave, with the received echoes being received at the separate receive locations of the receive elements R₁-R₅. FIG. 2d is another example of signal lead connections in which all of the transmit elements T₁-T₅ are driven simultaneously by a transmit signal on conductor 18a, and all of the receive elements R₁-R₅ are electrically coupled together and operated in tandem. All of the echo signals received by all of the receive elements R₁-R₅ at their respective positions are combined and conducted on the same conductor 18b. FIG. 2e is an example of a configuration in which each transmit element and each receive element can be operated individually. Each transmit element T₁-T₅ is coupled to its own signal conductor 18a and each receive element R₁-R₅ is coupled to its own signal conductor 18b. This example may be preferred when the sensor strip is operated by a battery-powered instrument, as only one transmit element is driven and only one receive channel is needed at any time, thereby conserving battery power.

FIG. 3a shows one example of how the transducer elements of a transducer pair may be positioned in the matrix 12 for improved signal reception. The Doppler ultrasound signal is angle-dependent. When the angle between the direction of the ultrasound beam and the direction of blood flow is 90°, the Doppler signal is at a minimum, and is strongest when the direction of blood flow is directly toward or away from the transducer. Since vessels close to the skin surface 30 such as the carotid artery 32, which is at an average depth in the body of 7 mm, are approximately parallel to the skin surface, a transducer orientation which transmits ultrasound waves normal to the skin surface 30 will have an angle of incidence of approximately 90° to the direction of flow. To reduce the probability of this orthogonal orientation the transducer elements are inclined at a shallow angle as shown in FIG. 3a. With the transmitting element T inclined as shown, it is seen that an obtuse angle L is formed between the direction 36 of wave travel and the blood flow direction 34 as indicated in FIG. 3b. In FIG. 3b the transducer elements T and R are offset from each other to enable them to be retained in the desired orientation by a matrix 12 of smaller thickness Th rather than the greater thickness of the matrix seen in FIG. 3a, thereby reducing the thickness of the sensor strip.

In the example of FIGS. 3a and 3b the element inclination angles the beam direction laterally with respect to the length dimension of the row of transducers, effectively causing the transducers to look to the side of the sensor strip. This works well when the sensor strip 10 is positioned across a blood vessel such as across the carotid artery 32 as illustrated in FIG. 4b. Positioning a sensor strip 10 across (orthogonal to) a blood vessel provides a layperson user with the greatest chance of intersecting the unseen vessel with ultrasound. The transmit transducer element aperture is thus looking toward or away from the direction of flow in the carotid artery 32. When the sensor strip is positioned as shown in FIG. 4b the strongest Doppler signal will be detected by the transducer pair T₃-R₃, which is positioned over the carotid artery 32 while the other transducer pairs are not over the vessel. In the Rock system shown in FIG. 4 of the '914 patent the row of transducers is aligned generally parallel to the length of the vessel. An advantage of this placement is that signals will be received by multiple transducer elements, increasing the signal to noise ratio, since multiple transducers are positioned over the blood vessel. A disadvantage is that, if the user misjudges the location of the blood vessel and positions the transducers parallel to but not over the hidden blood vessel, little or no signal will be received. The examples of FIGS. 3a, 3b and 4b will improve the likelihood of success for the layperson user.

FIG. 4a is a block diagram of a vital signs monitor and therapy system constructed in accordance with the principles of the present invention. A central processing and control unit 160 controls the various functions and components of the system and processes vital signs data. The central processing and control unit executes processing and control algorithms appropriate for the vital signs being monitored and the treatment being carried out by the system. The central processing and control unit may be connected to other devices by wired or wireless LAN connections or Bluetooth connectivity. The central processing and control unit 160 and other electronic components of the system are powered by a power subsystem 162 which may include a battery, a.c. line, power supply, and other power management and control functions. The clinician interacts with the system by means of a user interface 164 which may include elements such as a display, audio input and output, keypads, and a printer. The patient's ECG is monitored and processed by an ECG and processing subsystem 166 which can perform such functions as impedance, ventilation and arrhythmia analysis. The system includes elements for other vital signs measurement and processing 168 such as SPO2, ETCO2, IBP NIBP, and others. The system includes therapy functions 170 such as pacing and defibrillation, high voltage systems, and patient isolation. The performance of CPR is measured by a CPR measurement subsystem 180 as described more fully below.

FIG. 4b illustrates in block diagram form a portion of a vital signs monitor and therapy system which uses a sensor strip 10 to help guide the administration of CPR in accordance with the principles of the present invention. The sensor strip 10 in FIG. 4b is wired with the transmit elements T₁-T₅ connected in common and the receive elements R₁-R₅ with separate outputs as previously shown in FIG. 2c. The sensor strip 10 is connected to a defibrillator 110, one of the therapy functions 170, which includes the following elements shown in the drawing. A transmit generator 40 generates transmit waveforms for the transmit elements of the sensor strip. The transmit waveforms exhibit a nominal frequency in the range of 3-7 MHz and in this example have a nominal frequency of 5 MHz, which is typical for vascular ultrasound applications. The transmit waveforms are amplified by an amplifier 42 and applied to the transmit transducer elements T₁-T₅. The receive transducer elements R₁-R₅ are coupled to a multiplexer 44 which couples the signals received by one of the receive transducer elements to its output. The selected receive signal is amplified by a low noise amplifier 46 and filtered by an r.f. bandpass filter 48. The receive signal is mixed down to baseband by mixers 52 and 54 which are driven in quadrature by reference signals referenced to the transmit waveform. The demodulated quadrature signals are labeled as I and Q in the drawing and comprise quadrature detected components of the Doppler flow vector. The I and Q signals are filtered by lowpass filters 56 and 58 and then filtered by thump filters or wall filters 62 and 64, which pass the flow velocity components to the exclusion of DC (stationary tissue) components and components from the vessel walls. The filtered quadrature components are filtered by Doppler filters 66 and 68 and applied to the two inputs of a dual analog to digital converter 70 which digitizes the Doppler signals. The Doppler signals are translated to the Doppler spectrum by a fast Fourier transform (FFT) processor 72. FFT processing for Doppler signals is well known in the art with different implementations described for instance in “Discrete-Time Signal Processing,” by Oppenheim & Schafer (Prentice Hall, 1989). In a typical implementation consecutive overlapping sequences of Doppler samples are loaded into sliding sample window registers padded with zeroes and processed to produce Doppler frequency signals f_{D} in a Doppler spectrogram centered around zero (DC) and bounded by ±½ the Doppler sampling frequency determined by the transmission interval rate, which is generally in the kiloHertz range. If not done by the FFT processor the amplitude of the Doppler signals is detected by a detector 74 to produce power Doppler output signals.

The power Doppler signals are coupled to an analysis module 100, included in the CPR measurement subsystem 180, which can analyze the Doppler signals in various ways. In one example the multiplexer 44 selects the signal from a different receive transducer element every 10 msec as described in our U.S. patent application No. 60/583,966 filed Jun. 29, 2004 and now filed as international application IB 2005/052127, the contents of which is incorporated herein by reference. This polling sequence is shown in FIG. 6a. The multiplexer first selects the signal from element R₁ which is responsive to transmit signals from element T₁. After this first sampling period the multiplexer selects the signal from element R₂ which is responsive to transmit signals from element T₂. The multiplexer continues by selecting signals from elements T₃, T₄, and T₅, then repeats the sequence as illustrated at time t6 in FIG. 6a. During this time the analysis module 100 is looking for a strong power Doppler signal which exceeds a given threshold, such as a predetermined noise level. A valid power Doppler signal is recognized as one which exceeds the threshold by a given signal to noise ratio. In this example the defibrillator system is sampling the power Doppler signals while CPR is performed on the patient. When the rescuer compresses the chest of the patient an amount of blood is forced out of the heart and the pressure wave will emanate through the vascular system, generally causing a pulsatile flow of blood in the carotid artery. The onset of this blood flow is detected during the polling sequence and, when recognized as a valid power Doppler signal by the analysis module, the multiplexer stops polling and continuously couples the valid Doppler signal to the system. In this example the valid Doppler signal is detected by receive transducer element R₃ which is immediately above the carotid artery. The signals from receive element R₃ are then continuously sampled by the system as indicated by the T₃/R₃ period beginning at time t12 in FIG. 6b. The Doppler frequency f_{D} of the valid signal indicates the flow velocity and the peak signal indicates the maximum instantaneous flow rate caused by the CPR.

The sampling sequence effected by the multiplexer 44 may exhibit any of a number of variations. For instance, if the analysis module senses a decline in the strength of the power Doppler signal from a selected receive element, the multiplexer may be controlled to begin sampling the signals from the receive elements on either side of the selected element to try to find a stronger signal at an adjacent receive element. As FIG. 6b illustrates, at time t15 the signal from transducer element R₂ is sampled for a sampling period followed by the sampling of the signal from element R₄ during the next sampling period. If a stronger Doppler signal is not found at either of these transducer locations the multiplexer will return to sampling the signal from transducer element R₃ as shown at time t17. If multiple processing channels are available in a given device, multiple transducer elements can be monitored simultaneously and the strongest Doppler signal used for analysis.

In addition to detecting velocity the period of the Doppler waveform is sensed by detecting the recurring peak velocity over several chest compressions. The periodicity of this rate of recurrence indicates the rate of chest compressions during CPR. As a result of this analysis the rescuer is audibly and/or visually coached to administer CPR properly. For instance, a typical CPR protocol may call for the rescuer to administer 15 compressions at the rate of 100 compressions per minute. If the rate of recurrence sensed by the analysis module is less than this desired rate the analysis module will apply a signal to an audio synthesizer 102 or the display screen to issue a verbal “press faster” instruction. The audio synthesizer will produce an audio signal which is amplified by an amplifier 104 and applied to a loudspeaker 106 which audibly instructs the rescuer to “press faster.” The analysis module will also compare the peak velocity of blood flow during the compressions to a desired minimum blood flow velocity to be attained by each chest compression. For instance a typical peak velocity value is about 1 m/sec. The reference used by the analysis module may be less than this nominal rate and if the desired reference velocity is not being attained the analysis module can issue a “press harder” command through the audio synthesizer and loudspeaker of the user interface 164. A visual display such as a row of LEDs or a graphical display can illustrate visually the strength of the flow signal in absolute or relative terms and/or the position along the row of transducer sensors where the strongest flow signal has been detected.

In addition to detecting the peak velocity and period of the Doppler waves the analysis module may produce other measures of the sufficiency of the blood flow caused by the CPR compressions, such as mean velocity, volume flow rate, pulsation index, and flow index as described in our U.S. patent applications Nos. 60/609,676 filed Sep. 13, 2004, and 60/613,996 filed Sep. 28, 2004, the contents of which are hereby incorporated by reference.

The systems of FIGS. 4a and 4b have other sensors which may be used in combination with the Doppler flow sensor to judge the effectiveness of CPR. A compression pad 80 is shown in FIG. 4b which is placed on the patient's chest and against which the CPR compressions are applied. The compression pad includes a force sensor as shown in U.S. Pat. No. 6,351,671 or preferably an accelerometer as described in U.S. Pat. No. 6,306,107. Each time compression is applied to the pad 80 a signal is produced which is amplified by an amplifier 82 and detected by a detector 84. The detected chest compression signal is then used in combination with the information derived from the Doppler flow signals. For instance, each occurrence of a compression signal should correlate in time with the sensing of a valid Doppler flow signal by the sensor strip. Thus, the compression signal can be used to time gate the analysis of the Doppler signal or to correlate and confirm the rate of compression periodicity sensed by the analysis module. The ECG signal, when present, can also be used as a time gate. The amplitude of the force or twice-integrated acceleration signal is a measure of the compressive force or compression depth of the applied compression and can be used in deciding whether to issue a “press harder” or “press softer” command. For instance, while a low flow velocity or volume flow rate may indicate that the rescuer should press harder, the compression signal may show that the rescuer is already pressing as hard or as deep as is safely done on a patient. The analysis module may then withhold the “press harder” command in consideration of this compression information.

The system of FIG. 4b also has chest electrodes 92, 94 which are adhered to the patient's chest and used to sense the patient's ECG signals and thoracic bio-impedance, and to deliver a defibrillating shock. The ECG and impedance signals are processed by an ECG, impedance module 96 and coupled to the analysis module where they may be used to assist in CPR coaching. For instance, as explained in the '671 patent, the impedance signal will exhibit a change when the chest is compressed and again when the compressive force is relaxed. The times of occurrence of these impedance changes can be used to correlate with or time-gate the Doppler signal analysis to confirm or improve the detection of these signals and the appropriateness of CPR coaching commands.

FIG. 5 illustrates the outline of a patient and shows the defibrillator 110 with the proper placement of the sensor strip 10 on the neck across the carotid artery, the compression pad 80 in the center of the chest, and the electrodes 92,94 placed in the customary positions for defibrillation. It will be apparent to one skilled in the art that the analysis module can correlate or combine signals from all of these sensors to better produce coaching commands for CPR. It is also possible to combine the sensor strip 10 and the upper defibrillation electrode 92 into one electrode which is placed on the patient's neck as described in US patent publication 2003/0199929.

FIG. 7 illustrates a method of using a defibrillation system such as that shown in FIG. 4 which has a Doppler flow sensor 10 and a CPR compression pad 80. In this example the defibrillator will start the CPR coaching mode as shown at step 120. The defibrillator may be preset to automatically begin with a CPR interval or the defibrillator may go into a CPR mode when no shock is advised or after a shock has been administered. Analytical techniques are also known by which a defibrillator may make the mode change automatically after analyzing the ECG signal. For instance, the amplitude of ECG signals is known to correlate with the probability of successful defibrillation. Also a high rate of ECG complexes has been shown to relate to defibrillation success and an analysis has been proposed which performs a Fourier transform of the ECG data and looks at the median frequency of the transformed data to make this determination. Thus low amplitude and low rate ECG signals may be taken as an indication to begin CPR and cause the system to switch to the CPR coaching mode. In this example the defibrillator is equipped with a CPR metronome, which can be a simple tone or beep which recurs at the desired rate of CPR, e.g., 100 Hz. The rescuer is thus guided at step 122 to synchronize his or her compressions with the recurrence of this tone. When a compression is applied the analysis module will receive a depth or force signal from the compression pad at step 124 and, virtually simultaneously, a valid Doppler flow signal from the sensor strip 10 over the carotid artery at step 126. These signals are analyzed by the analysis module to determine the blood flow and its sufficiency in step 128. If the information indicates that a coaching instruction is needed an audible and/or visual coaching instruction or other feedback is issued at step 130. The system then returns to waiting for the signals of steps 124 and 126 at the time of the next chest compression.

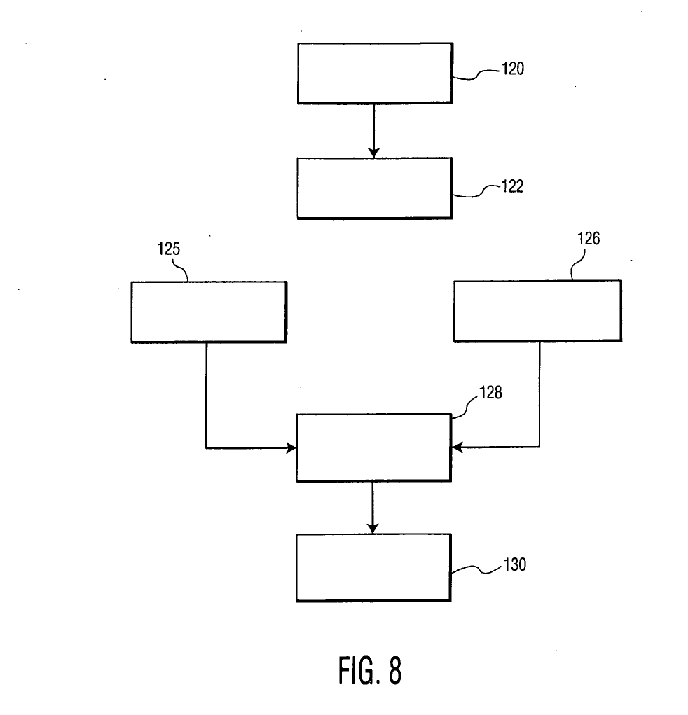

FIG. 8 shows a method with steps identical to those of FIG. 7, except that the compression signal is replaced by the impedance change signal in step 125. This information is used in conjunction with the Doppler flow signal to decide whether to issue a coaching instruction to guide the CPR.

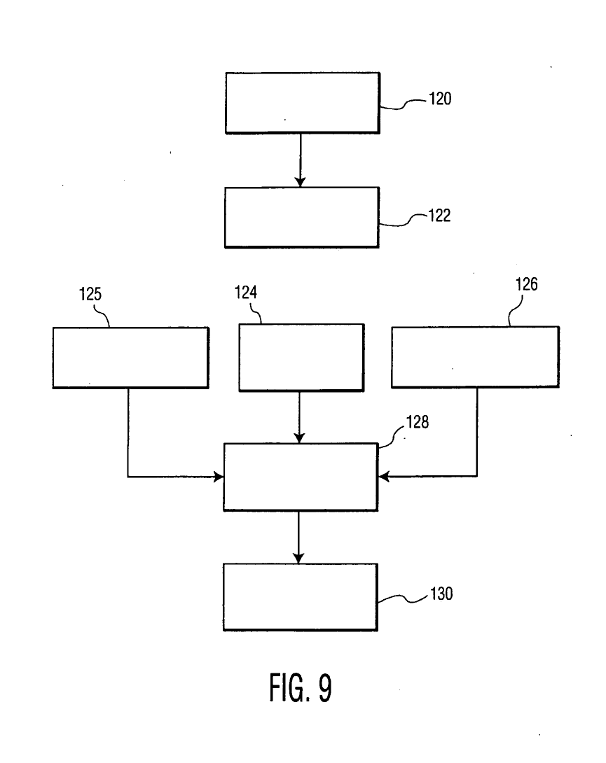

FIG. 9 combines the sensors of FIGS. 7 and 8 and uses information from all three sources to make determinations in guiding CPR. This method uses an impedance change signal in step 125, a compression signal in step 124, and a Doppler flow signal in step 126. As illustrated in the initial discussion of FIG. 4, the Doppler flow signal can be used without these other signals to guide effective CPR.

As previously mentioned the sensor strip in the previous examples is adhesively or mechanically attached to the neck of the patient over the carotid artery. It is important that a good acoustic coupling be established between the transducer elements and the skin surface for the reliable transmission and reception of ultrasound signals. This is generally provided by using a hydrophilic adhesive, an acoustic coupling gel over the skin surface opposed by the transducer elements, or a combination of the two. However the acoustic path can be disrupted if the sensor strip should loosen, which can occur through movement of the patient, perspiration or dirt on the skin surface which retards adhesive attachment, or drying out of the adhesive. When this occurs it is desirable to alert the rescuer or caregiver to the condition so that the problem can be corrected. In accordance with the principles of the present invention FIG. 10 illustrates an example in which the attachment of the sensor strip is monitored by a force sensor 150. In this example a force sensor 150 is located between the substrate 14 which in this example is an adhesive strip, and the matrix 12 containing the ultrasound transducers T and R. When held securely in place the matrix 12 is applying force to the skin surface 30, depressing the skin slightly as shown in the drawing. This downward pressure is applied by the tension of the attached adhesive strip 14. The forces involved are separately shown in FIG. 11. The force sensor measures the force F₁ applied by the tension T of the adhesive strip 14. The force sensor has a weight W_{FS} and the ultrasound transducer 1 has a weight W_{UT}. The ultrasound transducer 1 experiences forces F₁+W_{FS} on its upper surface. The skin surface 30 experiences the forces F₁+W_{FS}+W_{UT}. In a typical implementation the force applied by the adhesive strip 14 is much greater than the weights W_{FS} and W_{UT} of the force sensor 150 and the ultrasound transducer 1. Thus the force measurement F₁ produced by the force sensor 150 is a good approximation of the force applied to the skin by the attachment mechanism and weight of the device.

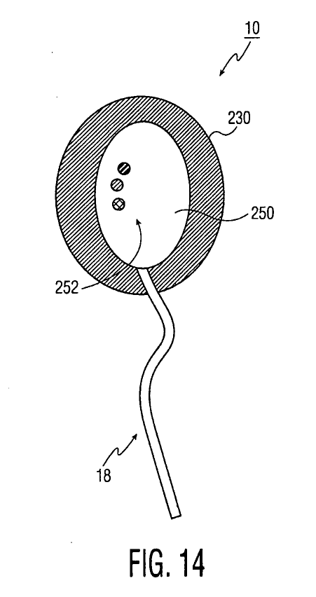

The force sensor may comprise any of a number of known sensor technologies. For instance the force sensor may comprise conductive rubber with electrodes embedded or located on each side of the rubber. The force sensor may be a piezoelectric sensor or it may be a strain gauge. Signals from the strain gauge can be conducted by wires contained in the cable 18 from which they are coupled to the defibrillator. A processor in the defibrillator monitors the force signal and if it drops below an acceptable level, an audible or visual alarm is issued. The alarm can issue from the AED or monitoring instrument to which the sensor strip is attached. In the example shown in FIG. 14 the alarm is issued from the sensor strip 10. In this example the transducers and force sensor are located in a housing 250 attached to an adhesive patch 230 which adheres the sensor strip to the body. The cable 18 connects the sensor strip to the AED or monitoring instrument. Located on the surface of the housing 250 are three different colored LEDs 252 which are illuminated while the sensor strip is attached to the body. If the force sensor is sensing that the sensor strip is in secure attachment to the body, the green LED next to the word “GOOD” is illuminated. If the force sensor senses no attachment force, the red “REDO” LED is illuminated. If the force sensor senses an ambivalent force which cannot be positively resolved as a secure attachment or not, the yellow “CHECK” LED is illuminated. The indicator signal may be derived from both the force signal of the force sensor and the ultrasound signal produced by the transducer: the force signal determines if the sensor is in good contact with the body and the lack of an acceptable ultrasound signal indicates that good acoustic contact with the body is no longer present. If conflicting signals were produced by the force sensor and the ultrasound transducer, for instance, the yellow LED could be illuminated. Instead of LEDs, an alphanumeric LCD display device can be used to convey quantitative information to the user if desired, such as the magnitude of the sensed attachment force. Alternatively or in addition to the visual indicator, an audible indicator may be employed, such as the piezoelectric speaker described in U.S. patent application Ser. No. 60/693,645, filed Jun. 24, 2005.

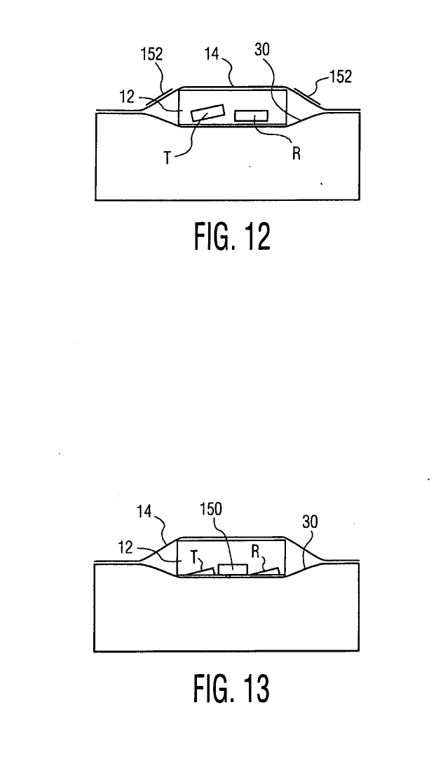

FIG. 12 illustrates another example of the use of a force sensor to measure the acoustic contact of the sensor strip 10. In this example the force sensors are strain gauges 152 on or in the adhesive strip 14 which may be above or on either side of the transducer elements as shown in the drawing. The signals from the strain gauges will indicate the tension of the adhesive strip when it is attached to the patient's skin and is monitored to assure that a desired level of tension is maintained while the sensor strip is in use.

FIG. 13 illustrates another example in which the force sensor 150 is contained in the matrix 12 adjacent to the transducer elements T and R. The advantage of this implementation is that the force of contact is measured directly adjacent to the point of transducer contact with the skin surface.

It will be appreciated that sensors measuring pressure rather than force may also be used in a constructed device of the present invention.

Another approach to monitoring the acoustic paths of the transducers is to measure the near field reflections from air pockets in the acoustic paths through signal processing. These air pockets will manifest themselves as strong near field echoes in the ultrasound signal. This can only be performed with a receiving transducer however.

What is claimed is:

1. An ultrasonic transducer assembly which is attachable to a body for the detection of motion or substances in the body comprising:
an ultrasonic transducer;
an attachment substrate connected to the ultrasonic transducer which acts to attach the ultrasonic transducer in contact with an acoustic path to the body; and
a force sensor positioned to receive the attachment force imparted by the attachment substrate and to produce a signal representative of the force of attachment.

2. The ultrasonic transducer assembly of claim 1, wherein the attachment substrate further comprises an adhesive substrate which acts to adhesively attach the ultrasonic transducer in acoustic contact with the body.

3. The ultrasonic transducer assembly of claim 1, wherein the attachment substrate further comprises a mechanical attachment substrate which acts to mechanically attach the ultrasonic transducer in acoustic contact with the body.

4. The ultrasonic transducer assembly of claim 3, wherein the mechanical attachment substrate further comprises an elastic band.

5. The ultrasonic transducer assembly of claim 3, wherein the mechanical attachment substrate further comprises a Velcro strap.

6. The ultrasonic transducer assembly of claim 3, wherein the mechanical attachment substrate further comprises a necklace.

7. The ultrasonic transducer assembly of claim 1, wherein the force sensor further comprises a piezoelectric sensor.

8. The ultrasonic transducer assembly of claim 1, wherein the force sensor further comprises a strain gauge.

9. The ultrasonic transducer assembly of claim 1, further comprising:
a monitoring instrument, coupled to receive the signal produced by the force sensor, and operable to produce an alert signal when the force signal is at an undesired level.

10. The ultrasonic transducer assembly of claim 9, wherein the ultrasonic transducer is operable to produce signals in response to blood flow; and
wherein the monitoring instrument is further operable to process the signals produced by the ultrasonic transducer.

11. The ultrasonic transducer assembly of claim 1, wherein the force sensor is positioned between the attachment substrate and the body when the assembly is attached to a body.

12. The ultrasonic transducer assembly of claim 1, wherein the force sensor is positioned on the attachment substrate.

13. The ultrasonic transducer assembly of claim 12, wherein the force sensor acts to sense the tension of the attachment substrate when the assembly is attached to a body.

14. The ultrasonic transducer assembly of claim 1, wherein the force sensor is positioned in the attachment substrate.

15. The ultrasonic transducer assembly of claim 14, wherein the force sensor acts to sense the tension of the attachment substrate when the assembly is attached to a body.

16. The ultrasonic transducer assembly of claim 1, wherein the force sensor is positioned adjacent to the body when the assembly is attached to a body.

17. The ultrasonic transducer assembly of claim 1, wherein the force sensor is positioned adjacent to the acoustic path of the ultrasonic transducer when the assembly is attached to a body.

18. An ultrasonic transducer assembly which is attachable to a body for the detection of motion or substances in the body comprising:
an ultrasonic transducer;
an attachment substrate connected to the ultrasonic transducer which acts to attach the ultrasonic transducer in contact with an acoustic path to the body;
a force sensor positioned to receive the attachment force imparted by the attachment substrate and to produce a signal representative of the force of attachment; and
an indicator, responsive to the signal representative of the force of attachment, which acts to alert a user to the state of attachment of the transducer assembly.

19. The ultrasonic transducer assembly of claim 18, wherein the indicator further comprises at least one of a visual indicator or an audible indicator.

20. The ultrasonic transducer assembly of claim 19, wherein the indicator is located on the ultrasonic transducer assembly when the transducer assembly is attached to a body.

21. The ultrasonic transducer assembly of claim 20, wherein the indicator comprises at least one LED indicator.

22. The ultrasonic transducer assembly of claim 18, wherein the ultrasonic transducer produces an ultrasound signal when attached to the body; and
wherein the indicator is further responsive to the signal representative of the force of attachment and the ultrasound signal.
